(19) United States
(12) Patent Application Publication
(10) Pub. No.: US 2003/0211458 A1
(43) Pub. Date: Nov. 13, 2003

(54) Measurements of enzymatic activity in a single, individual cell in population

(76) Inventors: Merav Sunray, Raanana (IL); Naomi Zurgil, Hertzlia (IL); Mordechai Deutsch, Lev HaSharon (IL)

Correspondence Address: John P White, Cooper & Dunham, 1185 Avenue of the Americas, New York, NY, 10036, US

(21) Appl. No.: 10/276,080

(22) PCT Filed: May 17, 2001
(86) PCT No.: PCT/IL01/00443

(30) Foreign Application Priority Data
May 18, 2000 (IL) 136232

(51) Int. Cl. C12Q 1/00; C12Q 1/54; C12Q 1/44; C12Q 1/37; C12Q 1/28

(52) U.S. Cl. 435/4; 435/14; 435/19; 435/23; 435/28

(57) ABSTRACT

A process for measuring enzymatic activity in an identified, isolated, intact, single, viable cell. Each of the viable cells is placed within individual identified locations on a carrier of a cytometer having means to measure enzymatic activity of a single viable cell placed in an identified location. The identified isolated cell is exposed to a substrate of an enzyme to be measured, and the rate of product formed or released following every exposure of the cell to same or different concentrations of the substrate is measured. The isolated cell may be exposed to a sequence of at least two different concentrations of the substrate, and for each exposure the rate of product formed or released, is measured.

### FIELD OF THE INVENTION

Enzymes are organic catalysts that cause and direct the numerous chemical reactions that occur in living organisms. Most of chemical changes that occur in living cells are caused and controlled by enzymes. Assessing the enzyme activity in a particular type of cells is therefore one of the principal approaches to the study of what goes on in the same individual living cells.

The present invention provides a new process and methodology for measuring enzymatic activity in intact individual cells. More specifically, it provides the capabilities for high precision enzymatic kinetic measurements of individual cells under repeatable substrate exposure conditions. On-line reagent addition, and controlling other changes in experimental conditions, can be easily accomplished, and the dynamic changes in individual given cells is monitored in real-time. Thus, the process of the invention provides a new valuable tool for assessing enzymatic reaction kinetics, resulting in determination of activity of an individual enzyme as well as of a series of different enzymes, in specific intact cells under defined physiologic conditions.

In a preferred embodiment of present invention, the substrate is either passively or actively enters the cell, once inside, it is processed by the assessed intracellular enzyme to generate detectable product.

In yet another preferred embodiment, the process of present invention is applicable for measuring simultaneously the enzymatic activity in many identified individual cells, within same population.

Since enzymes are ubiquitously involved in cellular function, the monitoring of their reaction kinetics on the level of a single, individual cell may provide valuable information. For example, in some human diseases, especially heritable genetic disorders, there may be a deficiency or even a total absence of one or more enzymes in the tissue. Moreover, measurements of the cellular activity of certain enzymes are important in diagnosing diseases. Most enzymes can be poisoned or inhibited by certain chemical reagents.

Numerous of drugs are designed to inhibit the excessive catalytic activity of specific enzymes in abnormal conditions. Other drugs inhibit certain enzymes in malfunctioning cells. The overall activity of such drugs can only be measured in an intact system of the individual live cell.

An enzymatic activity is usually characterized by two parameters: V_{MAX}—the maximum enzyme production rate (velocity) of a product (P) out of a substrate (S) at a saturation concentration of the latter, and K_{M}—the Michaelis-Menten constant, which is reciprocally proportional to the enzyme affinity to the substrate.

The relation between V_{MAX}, K_{M}, the substrate concentration [S] and the initial velocity V, at which S converts to P, is given by the Michaelis-Menten equation:

$$V = \frac{[ S ] \cdot {V}_{MAX}}{{K}_{M} + [ S ]} .$$

Unfortunately Eq. 1 is accurate only for a homogeneous medium in which the following processes occur: [S]+[E]⇄[ES] and [ES]→[P]+[E] where [E] and [ES] are the enzyme and the complex enzyme—substrate concentrations, correspondingly.

The determination of K_{M} and V_{MAX}, utilizing Eq. 1 calls for sequential exposures and repeatable measurements of the same individual cell for various values of [S].

Unfortunately this requirement can not be achieved by the common cytometers: The Flow Cytometer (FC) as well as the Laser Scanning Cytometer (LSC). The FC enables the rapid measurement of the fluorescence intensity (FI) of a large cell population. However because each cell in the flow is measured only once, the kinetic curves of the FC.

1. Dolbcare F, Fluorescent staining of enzymes for flow cytometry, Methods Cell Biol 33:81-88, 1990

2. Klingel S, Rothe G, Kellerman W, Valet G, Flow cytometric determination of serine proteinase activities in living cells with rhodamine 110 substrates, Methods Cell Biol 41:449-460, 1994

3. Malin-Berdel J, Valet G, Flow cytometric determination of esterase and phosphatase activities and kinetics in hematopoietic cells with fluorogenic substrates, Cytometry 1:222-228, 1980

4. Nooter K, Herweijer H, Jonker R R, van den Engh G J, On-line flow cytometry. A versatile method for kinetic measurement, Methods Cell Biol 41:509-526, 1994

5. Turck J J, Robinson J P, Leucine aminopeptidase activity by flow cytometry, Methods Cell Biol 41:461-468, 1994

6. Watson J V, Dive C, Enzyme kinetics, Methods Cell Biol 41:469-508, 1994] provide sequential measurements of single cells over time but not of the same single cell. Therefore, investigating different enzyme activities in different cell types or in subcellular areas using the FC gives only an average K_{M} value for a population of cells or for specific enzymes in a cell-free system.

The LSC measures the fluorescence kinetic of individual cells under specific conditions of low cell density in the selected field and of cell types and dyes which do not suffer from fading, which disrupts the measurement [Watson J V, and Dive C. Enzyme kinetics. Methods Cell Biol (1994) 41:469-508]. The LSC technique cannot ensure the accurate rescanning of the same cell after repeatable staining procedures since the cell may not have preserve its original location. Moreover, the LSC cannot ensure preservation of the cell locations and thus cell identification might be lost during repeatable rinsing and exposure to different substrate concentrations.

In order to provide the capabilities for kinetic measurement of individual cells under repeatable staining conditions, a specially designed cytometer was used. The cytometer (hereinafter referred to as Cellscan Mark S or CS-S) which, one of its versions, was described in the U.S. Pat. Nos. 4,729,949, 5,272,081, 5,310,674 and 5,506,141 found to be applicable for measuring time resolved kinetics of individual cells during cellular manipulation.

Using the unique application of the CS-S, a new method was developed in which the same cells are sequentially exposed to increasing substrate concentrations. The product formation rate is measured for each cell at every substrate concentration yielding a series of rates for the same individual cell. Using this data, V_{MAX} and apparent K_{MAPP} (app=apparent) values can be calculated for each cell, giving the distribution of K_{MAPP} and V_{MAX} of the measured population However, it should be emphasized that the process of present invention is not limited to the CS-S cytometer and any cytometer comprising a microscope, light detection means, a carrier to which cells are individually located, is within the scope of the present invention.

Kinetic Analysis:

The kinetic parameters are derived by application of linear and nonlinear modeling. The linear model y(t)=At+B seeks parameters A and B which fit the data to a straight line equation, where y(t) is the measured quantity, t is the time, and A and B are the calculated parameters. The CS-S algorithm uses χ² as the criteria for goodness-of-fit.

a. Single Step Cell Staining:

A simplified model for the description of intracellular turnover of fluorogenic substrate is presented in FIG. 1. First, the extracellular substrate [S]ₒ permeates into the cell, becoming [S]i—the intracellular substrate concentration. Then [S]i is hydrolyzed or cleaved by enzymes to yield the intracellular (for example, fluorescent) product [P]i, which may be released from the cell into the medium and become [P]o.

As was previously shown [Bedner E, Melamed M R, Darzynkiewicz Z, Enzyme kinetic reactions and fluorochrome uptake rates measured in individual cells by laser scanning cytometry, Cytometry 33:1-9, 1998] the kinetics of [P]i can be described, to a good approximation, by the rate equation:

$$\frac{ⅆ {[ P ]}_{i}}{ⅆ t} = α \cdot {[ S ]}_{O} - β \cdot {[ P ]}_{i} \qquad ( 2 )$$

Where α and β are the rates constants for the formation and leakage of the intracellular fluorescein. It is important to emphasize that a represents two processes: Permeation of S and its intracellular distribution as well as the enzymatic hydrolysis of [S]i.

When solving Eq. 2, under the initial condition of one step staining, [P(t=0)]_{I}=0 it is easily shown that

$$P ( t ) = {\frac{α}{β} [ S ]}_{O} ( 1 - {ⅇ}^{- β \cdot t} ) \qquad ( 3 )$$

b. Sequential Staining:

Another aspect of present invention relates to sequential exposures of the same individual cells to different substrate concentrations. This differs from the above case by the fact that at the starting time point of staining, with a given solution, cells are already being stained to a level of:

$${[ P ( τ ) ]}_{i} = \frac{α}{β} M [ S ] ( 1 - {ⅇ}^{- β τ} ) \qquad ( 4 )$$

τ stands for the time point of terminating the staining with a given substrate concentration, say M times [S] (M[S]),and initiation of staining with different substrate concentration, say N[S].

Now, it is possible to solve Eq.2 under the initial conditions presented by Eq. 4. By separation of variables and integration over [P]ᵢ between the concentration limits [P(τ)]ᵢ and [P(t)]ᵢ; and integration over time between the time points 0 (when staining solutions are being replaced) and t, one gets:

$${\int }_{{[ F ( τ ) ]}_{I}}^{{[ F ( t ) ]}_{I}} \frac{{d [ F ]}_{I}}{{[ F ]}_{I} - \frac{α}{β} N [ S ]} = {\int }_{0}^{t} - β ⅆ t ⇒ ln ( \frac{{[ F ( t ) ]}_{I} - \frac{α}{β} N [ S ]}{{[ F ( τ ) ]}_{I} - \frac{α}{β} N [ S ]} ] = - β \cdot t \qquad \underline{( 5 )}$$

Converting the logarithmic expression into exponential one and introducing [F(τ)]_{I} of Eq.4 into Eq.5 yields:

$${[ F ( t ) ]}_{I} = \frac{α}{β} M [ S ] ( 1 - {ⅇ}^{- β τ} ) {ⅇ}^{- β t} + \frac{α}{β} N [ S ] ( 1 - {ⅇ}^{- β t} ) \qquad ( 6 )$$

When single step staining is performed (starting of unstained cell, M=0), only the last term of Eq. 6 remains, which is consistent with Eq. 3.

As long as the expression exp(−βt)≅1−βt holds for the duration of the observation interval of the individual cells in given conditions, regardless of their staining history, each of the exponential terms in Eq. 6 can be replaced, without losing accuracy, by its first two terms of the power series. Hence, Eq. 6 may be linearly approximated to give:

$${[ F ( t ) ]}_{I} = \{ \begin{matrix}0 < t < τ \\ t > τ\end{matrix} \to \begin{matrix}α [ S ] M t \\ α [ S ] \cdot ( M τ + N t )\end{matrix} \to \begin{matrix}\frac{ⅆ {[ F ( t ) ]}_{I}}{ⅆ t} = α [ S ] M \\ \frac{ⅆ {[ F ( t ) ]}_{I}}{ⅆ t} = α [ S ] N\end{matrix} \} \qquad ( 7 )$$

Eq. 7 should be interpreted as follows: for 0<t<τ, staining proceeds according to [P(t)]ᵢ=α[S]Mt. After replacing the staining solution M by N at time t=τ, the staining due to M[S] remain constant (P(τ)_{I}=α[S]W_{τ}, While that due to N increases at a rate of α[S]N, namely solely depending on the concentration in use. Simulations of several practical staining protocols, based on Eq.7, are graphically presented in FIG. 2 and briefly described in the following:

a) Rinsing the cells with a staining solution [N] that maintains [N]=[M], results in a staining curve [P(t)]_{I}=α[S]N[τ+t]. At the observation time τ+t [P]ᵢ had a production rate of α[S]N, the same rate as that of α[S]M prior to τ+t (FIG. 2a).

b) Rinsing the cells with PBS alone washed away [M] residues leaving the staining solution at a concentration [M]=0. This action halted any further production [P]_{I} (since α[S]N=0 at the time of application τ) hence [P]ᵢ line remained parallel to the time axis for the duration of the observation t. (FIG. 2b).

c) In a similar way, the cells were rinsed with a staining solution [N]≠[M] that washed away [M] and left the staining solution at a concentration [N]. The production rate of [P]ᵢ, as expected, changed to α N [S] for the observation duration t. (FIG. 2c)

d) The last experiment, was a combination of b) and c) in succession. First the cells were rinsed at time t₁ with PBS and that halted the production of F_{I}. The next stage was to rinse with a staining solution [N]≠[M] replacing the PBS with a solution of concentration [N]. The production rate then changed to α[S]N for the for the observation duration t. (FIG. 2d).

Finally, the determination of Δt, the overall sequential staining experiment procedure time duration, was restricted to follow the present CS-S Standard deviation in performing individual cell FI measurements, which is <2%.

In order not to exceed this value when linearly approximating the exponential terms, a Δt value which keeps the ratio

exp(−βΔt)/(1−βΔt)≡2% is sought. Hence, introducing β≅10⁻⁴ sec⁻¹, which is the outcome of many hundreds of independent experiments (data not shown), yields Δt≅103 sec.

### SUMMARY OF THE INVENTION

It is an object of present invention to provide a process for measurement and characterization of intra- and extra-cellular enzymatic activity taking place in the same identified individual cell, in a population of cells, following its incubation with different concentrations of a substrate. The substrate should be a substance that yields a product that is detectable by physical means, such as changes in fluorescence intensity, color intensity, radioactive radiation, etc.

It is a further object of present invention to establish a new method for the determination of K_{MAPP} and V_{MAX} values for enzymatic reactions carried out inside an identified individual cell. It is an additional object of the present invention to determine kinetic values of extracellular enzymes, released from an individual cell. It is yet an additional object of present invention to provide a tool for measuring differences in kinetic enzymatic activity in the individual cell following various treatments of same cell with biologically active materials.

A further object of present invention is to provide a process for measuring simultaneously the enzymatic activity in many identified individual cells, within same population.

### BRIEF DESCRIPTION OF THE DRAWINGS

FIG. 1: A model of intracellular conversion of a substrate to a product. [S]₀, [S]ᵢ are the extracellular and intracellular substrate concentrations and [P]₀, [P]_{I} are the extracellular and intracellular product concentrations. [E] and [ES] are the enzyme and enzyme substrate complex concentrations. k₁ is the rate constant for formation of the complex [ES], k₁ is the rate constant for the reversed reaction and k₂ is the rate constant for product formation.

FIG. 2: Simulation of an individual cell sequential FI time dependency following several exposure procedures to substrate concentration. M=multiplication coefficients of initial substrate concentration.

R=rinsing at a given time point. Panels: a—rinsing with the same concentration yields identical slopes. b—sequential rinsing with (yield identical slopes as in panel a) and without (zero slopes) substrate. c—sequential rinsing with increasing substrate concentrations. d—sequential rinsing with increasing substrate concentrations while in between rinsing without substrate.

FIG. 3: Experimental results of individual cells sequential staining procedure. The numbers in the boxes are the slopes of FI(t) (initial velocities), given in arbitrary intensity units per second. The experiment follows the simulation shown in FIG. 2.

FIG. 4: Complete sequential staining procedure of numerous cells. Each of the four clusters contains 13 lines. Each line defined by six FI measurements taken in six different time points for the same individual cell when exposed to the relevant substrate concentration. R₁ to R₄—the space between clusters stands for replacement duration of the staining solutions (0.6, 1.2, 2.4 and 3.6. μM). The solid line in each of the four clusters is sketched for clarification purposes. It indicates the increasing slopes of one chosen set of sequential exposure of one individual cell.

FIG. 5: Individual K_{MAPP} and V_{MAX} for two representative cells and their Pearson correlation coefficient (R²).

FIG. 6: The distribution of individual K_{MAPP} (6A) and V_{MAX} (6B) for cells that were incubated with (-) and without (- - -) PHA.

FIG. 7: Rate of change of FI before and after exposure of an individual cell to hydrogen peroxide (H₂O₂) compared with control. The ratio pre to post treatment slopes in control cells is double that of cells exposed to H₂O₂ (treated).

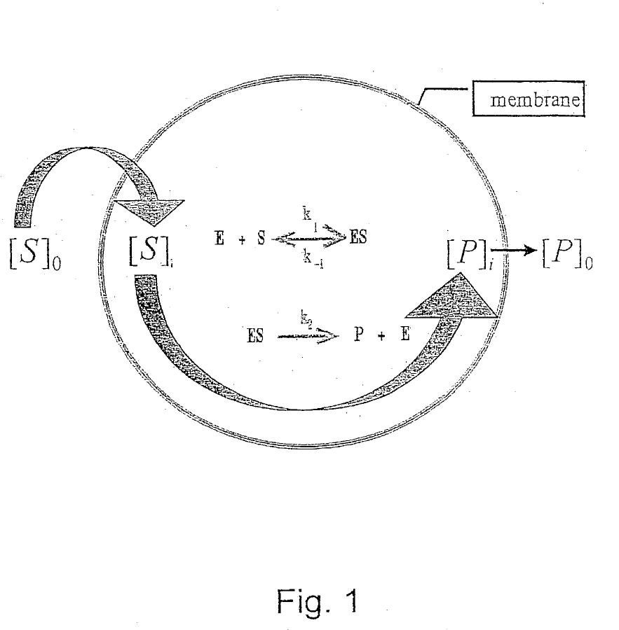

The following examples are provided merely to illustrate the invention and are not intended to limit the scope of the invention in any manner.

### EXAMPLES

### Example 1.

Measuring Intracellular Nonspecific Esterase Activity in a Single Lymphocyte Using Fluorescein-Diacetate (FDA) as the Substrate.

Materials and Methods

Phytohemagglutinin PHA (HA15, Murex Biotech) was reconstituted in 5 ml of double-distilled water and further diluted ten times. For stimulation, 10 μl of this solution was added to a 90 μl cell suspension (7×10⁶ cells/ml).

The culture medium consisted of RPMI-1640 (Biological Industries), supplemented with 10% (v/v) heat-inactivated fetal calf serum (Biological Industries), 2 mM L-glutamine, 10 mM Hepes buffer solution, 1 mM sodium pyruvate, 50 U/ml penicillin and 50 Units/ml streptomycin.

A staining solution of 3.6 μM FDA (Riedel-de Haen Ag. Seelze-Hanover) in Dulbecco Phosphate Buffered Saline (PBS, Biological Industries) was prepared as follows: 50 mg of FDA was dissolved in 5 ml of DMSO (Sigma). 7.5 μl of this solution was added to 50 ml PBS. For 0.6, 1.2 and 2.4 μM the solution was further diluted in PBS.

Preparation of Peripheral Blood Mononuclear Cells (PBMC):

30 Heparinized blood (30 ml), was taken from healthy, normal volunteers. The procedure for separating the PBMC has been described in detail, elsewhere [Sunray M, Deutsch M, Kaufman M, Tirosh R, Weinreb A, and Rachmani H. Cell Activation influences cell staining linetics, Spectrochimica Acta A (1997) 53:1645-1653]. Shortly after removing the iron absorbing cells, the remaining cells are layered on a two-layer (100% and 80%) cell density gradient (Ficoll Paque, Pharmacia 1.077 g/ml) and centrifuged. The cells accumulated at the interface between the two Ficoll layers, were collected and kept at 37° C. in 5 ml of enriched culture medium overnight. The next day the PBMC were washed and resuspended in PBS at a final concentration of 7·10⁶ cells/ml. More than 70% of the cells were defined as T lymphocytes and viability, which was determined using eosin, was always higher than 90%.

Activation of PBMC by PHA:

Freshly prepared PBMC (7·10⁶ cells/ml) were incubated at 37° C., 5% CO2 with 5 μgr/ml PHA for 30 minutes. PBMC controls were incubated without PHA under identical conditions.

The CS-S Apparatus

The multiparametric, computerized, discrete cytometer CS-S used in performing this example was described in detail in the above specified US Patents. Its central feature is a cell carrier (CC) incorporating a 100×100 dimensional array having a conical cross-section with an upper opening of ˜7 μm and lower opening of ·4 μm, each approximately 20 μm apart, in which individual cells are trapped. The cell carrier is mounted on a computer-controlled stage that enables repeated multi-scanning of the same cells.

Cells were irradiated with 1-10 μW of 442 nm light from a He—Cd laser. Under the staining conditions used here, the scanning time for obtaining a count of 10,000 photons in order to have statistical photonic error of ˜1% from each, dye-loaded cell varied from 0.001 sec to approximately 0.5 sec.

The acquired data, including cell position, measurement duration for each cell, absolute time, intensity at two different wavelengths, computed fluorescence polarization values and test set-up information, are displayed on the screen, on-line, graphically and numerically, and stored in the memory. Software enables the determination of the range and other statistical characteristics of all parameters, for either the entire cell population, or an operator-selected sub-population, or an individual cell, before, or during the scan.

Cell Loading

Loading the cells in wells traps on the Cell Carrier (CC) was carried out, as described in Deutsch M, and Weinreb A., Apparatus for High Precision Repetitive Sequential Optical Measurement of Living Cells, Cytometry (1994) 16: 214-226. An aliquot of 80 μl of unstained cell suspension (7×106 cells/ml) was loaded on the CC. Initial scanning was then performed in order to detect individual cell background scattering and auto-fluorescence. This undesired signal is recorded per measurement location and subtracted from the total emission signal (after exposure) in order to obtain the correct fluorescence signal.

Cell Staining and Kinetic Measurement:

For fluorescence intensity FI(t) measurements, trapped cells on the CC were sequentially exposed to increasing concentrations of FDA in PBS staining solutions.

Following background measurement, the volume of PBS, which covers the cells, was pumped out and the following procedure was carried out:

At time point zero, 40 μl of the lowest substrate concentration solution was applied on top of the trapped cells and a pre-chosen cell field was sequentially scanned 6 times. This yielded 6 accurately timed FI data points per each individual cell at a given dye concentration. FI is usually measured utilizing epi-fluoescence optical arrangement which permits the differentiation between the excitation energy and the emitted fluorescence energy to be detected by photomultipliers, CCD detectors etc.

The above procedure is repeated for each different substrate solution used in the experiment.

This yielded six FI data points for each individual cell, per substrate concentration, from which V was extracted and the individual cell K_{MAPP} and V_{MAX} values were calculated. The dead time, i.e., the elapsed time from the addition of a staining solution to the beginning of the measurement, which is monitored by the computer, is about 7-15 sec.

Results

Repeatability Runs:

The experimental arrangement of the new process calls for high-level performance in terms of repeatability and accuracy in periodical measurements of individual cells.

The CS-S capability was displayed by performing sequential measurements of FI and FP on 5 min 1.2 μM FDA stained trapped cells, following their PBS rinsing out of excess substrate solution and possible extra-cellular P_{I} (at this stage, constancy of FI is expected due to staining termination and negligibility of P_{I} leakage).

The individual cell coefficient of variance (CV) obtained in more then 10 successive measurement scans of a 10×10 cell field, never exceeded 2% for FI. Fading was not noticeable.

Accuracy Runs:

Accurate intensity measurement capabilities and specific monitoring of alterations in FI production rate are mandatory for the present process. This was first serially examined by measuring FI of the CC loaded with cell-free fluorescein solutions at concentrations of 0.6, 1.2 and 2.4 μM, five times each, while rinsing with PBS between concentrations.

The ratios, FI([s]ᵢ)/FI([s]ⱼ), between the measured FI, for different [s] and fluorescein concentrations, were found to be in correlation to the ratios of FDA substrate concentrations ([s]_{I}/[s]_{J}) and free fluorescein concentrations ([F]ᵢ/[F]ⱼ), (see Table 1).

The correlation between the substrate concentration (FDA concentration) and the measured staining rates by intracellular fluorescein was established for cells. First, each CC was loaded with unstained (BPS free of substrate) cells and stained with one chosen substrate concentrations (in order to avoid possible influences of additive staining when sequential exposure is performed).

Second the sequential staining manipulation was examined. As can be seen in the third and forth column of Table 1, there was good correlation between the increasing staining rates (which means increasing rate of product formed) and the increasing substrate concentrations in both cases.

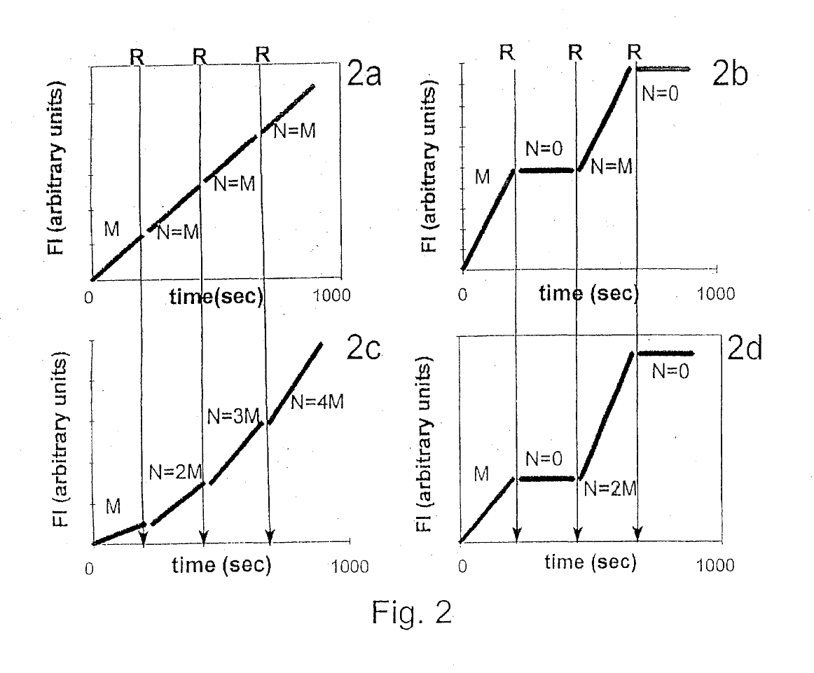

Next, using the sequential staining manipulation [adding in sequence of different concentrations of substrate and measuring the production rate of F in between additions, by monitoring FI(t)] with cells, it was verified the theory described in equation 7 specifically for the four cases that are detailed above and are presented as simulations in FIG. 2.

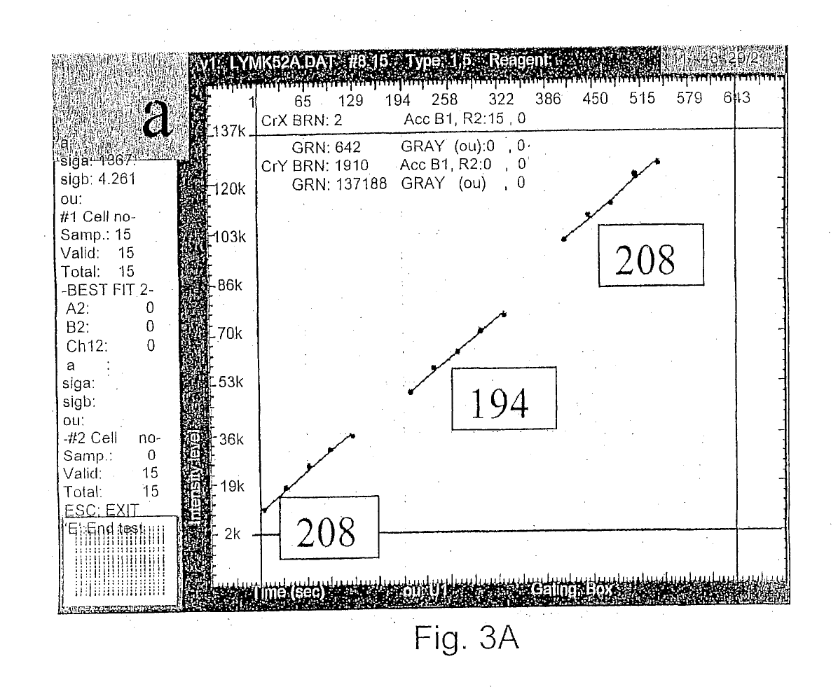

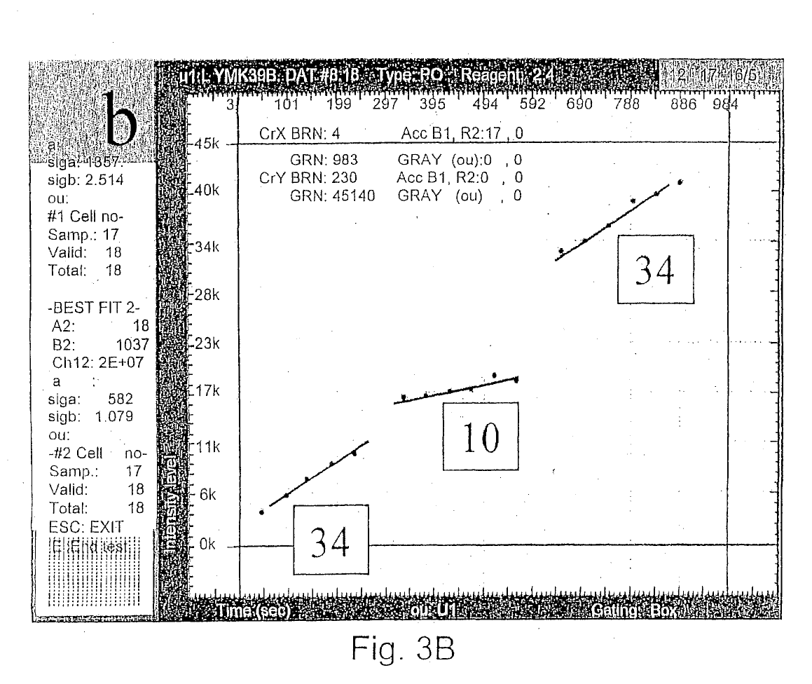

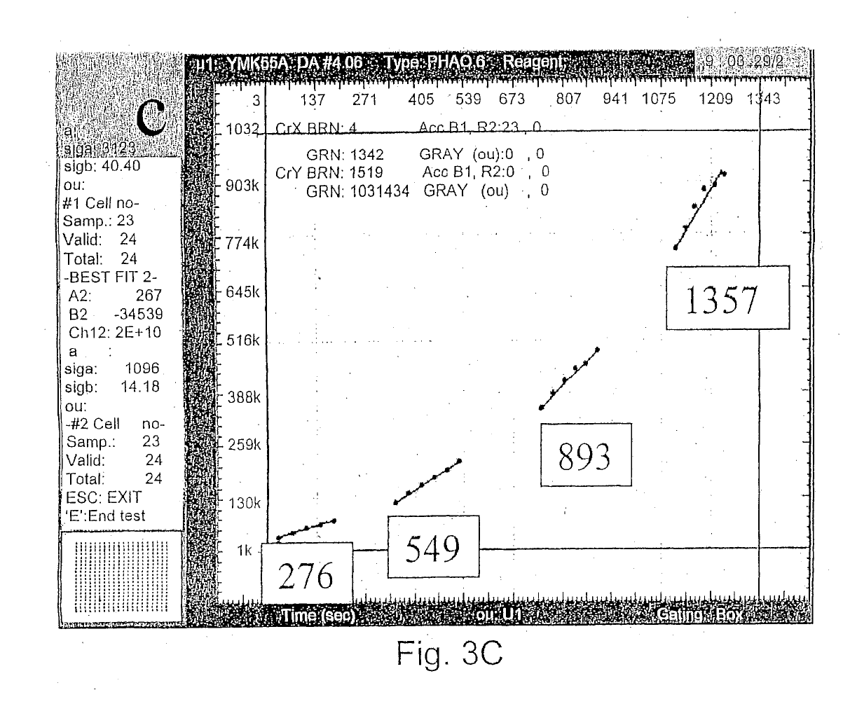

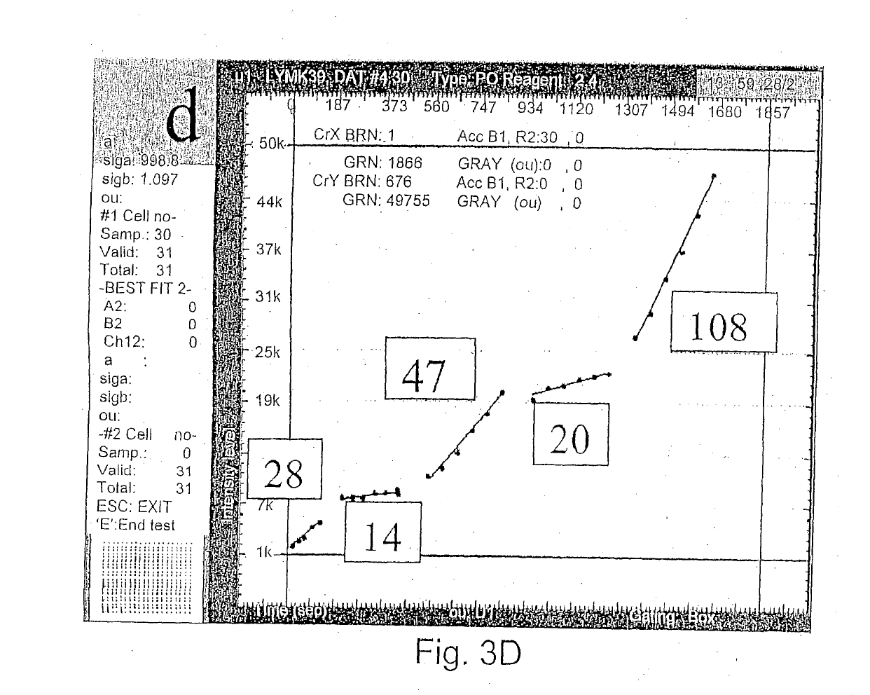

First, cells were loaded on the CC and stained with FDA. Re-washing the cells after every five or six scans with the same FDA concentration gave similar slopes after every wash as can be seen for FDA at 1.5 μM in FIG. 3a.

Rinsing (R) the cells with FDA and with PBS (no FDA, N=0, equation 7) alternately gave similar slopes when FDA was present and almost zero slope when FDA was absent, as shown in FIG. 3b.

The level of FI at the be,inning of the last rinse was higher than the level at the end of the rinse with PBS though the slopes (velocities which are the magnitude used for K_{MAPP} determination) were identical. This difference is probably due to technical reasons such as a slight change in the focus while manipulating FDA concentration or by laser beam geometrical instability etc. In FIG. 3c the cells were rinsed with increasing FDA concentration of 0.6, 1.2, 2.4 and 3.6 μM.

In FIG. 3d, the cells were rinsed with FDA at concentration of 0.6, 1.2, 2.4 μM and in between with PBS without FDA. The PBS gave almost zero slopes (no production of FI) while the increasing FI slopes were in good correlation with the increasing FDA concentration. Generally, as can be seen from FIGS. 2 and 3, there was good correlation between the theoretical simulation and results of the experiments.

TABLE 1

| | | FI rate ratios | FI rate ratios of |
|---|---|---|---|
| | | of cells parallely | the |
| Substrate | Ratios of FI of | exposed to different | same trapped |
| concentrations | various | FDA concentrations | cells sequentially |
| [s] ratio | fluorescein | cell | different FDA |
| (μM) | solutions | Carriers | concentrations |
| (a) | (b) | (c) | (d) |
| 2.0 1.2/0.6 | 2.1 | 1.9 | 2.1 |
| 4.0 2.4/0.6 | 4.0 | 4.4 | 3.6 |
| 6.0 3.6/0.6 | — | 6.2 | 5.6 |

Table 1: Ratios of substrate concentrations (a); of fluorescein solutions FI (b); and of intracellular fluorescein production rates of trapped cells; (c) parallely exposed on different CC each to different FDA concentrations and (d) sequentially exposed to different FDA concentrations on the same CC.

Determination of Individual K_{MAPP} and V_{MAX} Values:

Determination of K_{MAPP} and V_{MAX} was carried out by utilizing the reciprocal of Eq. 8 (Lineweaver—Buxk plot)

$$\frac{1}{V} = \frac{{K}_{M}}{{V}_{MAX}} \frac{1}{[ S ]} + \frac{1}{{V}_{MAX}} \qquad ( 8 )$$

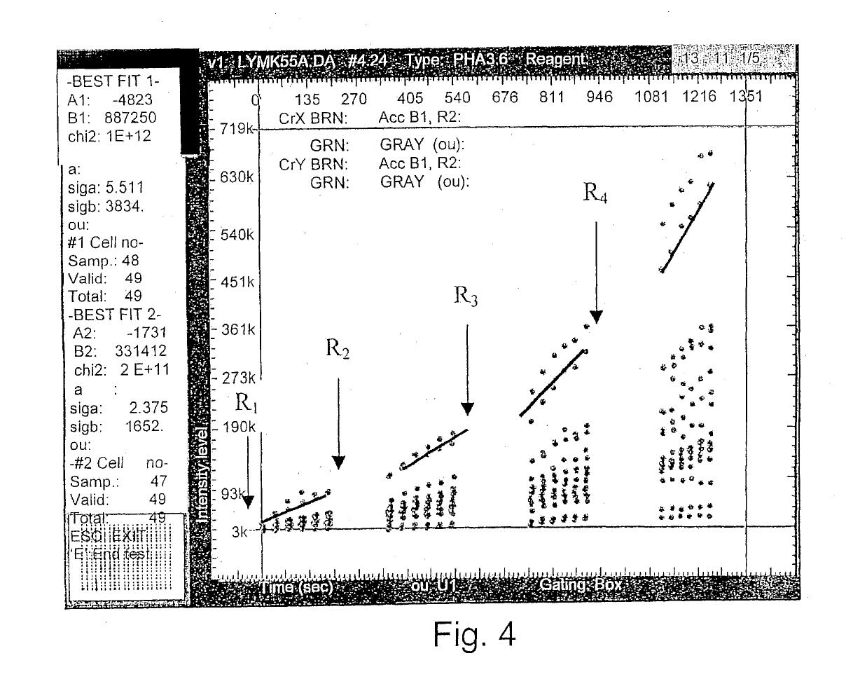

Thus, the use of two substrate concentrations should be, in principal, enough for the extraction of K_{M} and V_{MAX}. Minimization of possible experimental errors, while restricted by the linear range of time duration, Δt≅10³ sec, led to the choice of 4 FDA concentrations: 0.6, 1.2, 2.4 and 3.6 μM. Practically, trapped cells on the cell carrier were sequentially exposed to the four FDA concentrations staining solutions and scanned for determination of released fluorescein six times per the same FDA concentration. A representative chart of a complete measurement procedure made on 50 cells is shown in FIG. 4.

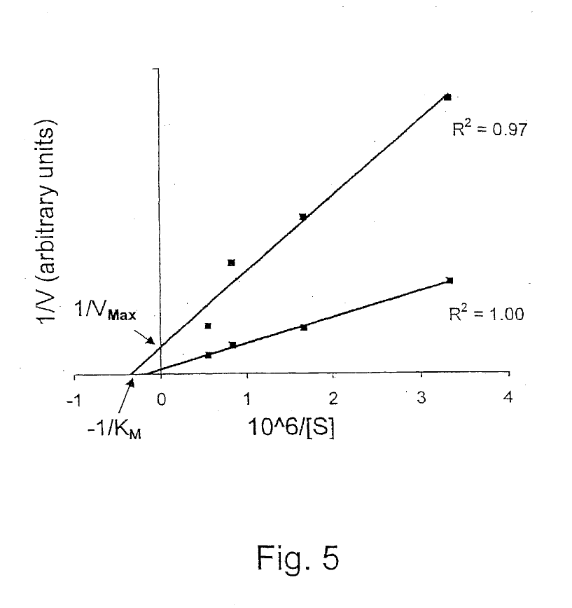

A plot of Eq. 8 for two cells out of the measured population of FIG. 4 is presented in FIG. 5.

### Example 2

Utilization of Individual K_{MAPP} Measurements

The Influence of Mitogenic Stimulation upon K_{MAPP} and V_{MAX}: The activation of lymphocytes is a critical stage in most immune responses and allows these cells to exert their specific functional capabilities. During activation, the resting lymphocytes undergo complex changes resulting in cell differentiation and proliferation. Lymphocytes activation is triggered by multiple interactions that occur at the cell surface, which initiate intracellular biochemical events within the cell that culminate in cellular response.

One of the experimental models used-to study lymphocytes activation is lectins, plant derived proteins (including phytohemagglutinin PHA), that bind carbohydrate groups at the cell surface and stimulate relevant receptors involved in physiologic lymphocyte activation. Many pharmacological agents mimic or inhibit some of the intracellular events associated with T cell activation. An example is described herein for individual K_{MAPP} measurement following lymphocyte activation.

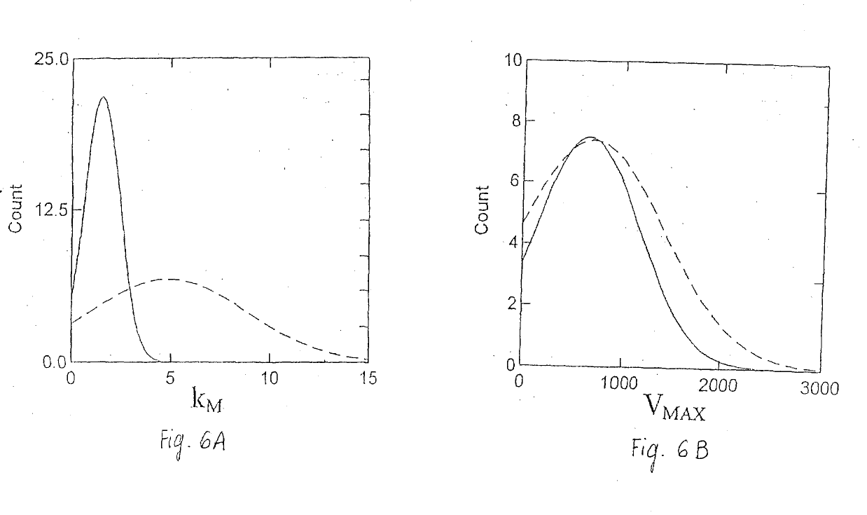

The sequential FDA hydrolysis experiment was executed following incubation of cells with and without phytohemagglutinin PHA. The distribution of individual K_{MAPP} and V_{MAX} values for both cases are presented in FIG. 6a and 6b, respectively. The average K_{MAPP} and V_{MAX} were found to be 4.88 μM and 1.50 μM and 695 (intensity/sec) and 652 (intensity/sec), indicating a decrease of 69% in K_{MAPP} and 6% in V_{MAX} values for PHA compared to the control. Both distributions indicated cell heterogeneity having a CV of about 70%.

For comparison purposes, at the average level, the FC (Beckton-Dickinson FACSCalibur) was used to determine K_{MAPP} and V_{MAX} value averages taken over the cell population following the protocol suggested by Watson, J. V and Dive, C., Enzyme Kinetics. Methods Cell Biol (1994) 41: 469-508. Four means of intracellular fluorescence intensities (IFI) were calculated from data accumulated along four time gates of 25 second each and 30 seconds apart, from which V₀ was extracted. This process was sequentially performed on five different aliquots of cells (50 μl, at a concentration of 6×10⁶ cells/ml) each exposed to different FDA concentrations (0.3, 0.6, 1.2, 1.8 and 2.4 μM. Introducing these average V₀ in values Eq. 8 yielded population average K_{MAPP} and V_{MAX} of 2.16 μM and 4.32 μM and 6.6 and 5.83 in cells incubated with and without PHA. It should be noted that while K_{MAPP} is an intrinsic value, V_{MAX} depends on the optoelelctronic arrangement under use. Thus, obviously, at the population level, measurements carried out both on FC and average calculated from individual cell K_{MAPP} measurement data yield similar K_{MAPP} values, indicating the validity of the invented methodology.

### Example 3

Using basically the same procedure, it is possible to determine the following enzymes activity in the single, individual cell:

1. Proteases and Peptidases

Peptidases and proteases play essential roles in protein activation, cell regulation and signaling, as well as in the generation of amino acids for protein synthesis or utilization in other metabolic pathways. Typical peptidase substrates are short peptides conjugated to fluorophores (like 7-Amino-4-methylcoumarin (AMC) or Rhodamine 110). In the presence of the enzyme, the fluorogenic part is released, and may be easily determined by fluorescence measurements. One example of peptidase is the cystein protease—Caspase which play a pivotal role in programmed cell death.

AMC- and R110-labeled peptidase substrates, permit the detection of apoptosis by assaying for increases in caspase-3 and caspase-3-like protease activities. The activation of caspase-3 (CPP32/apopain), which has a substrate selectivity for the amino acid sequence Asp-Glu-Val-Asp (DEVD) and cleaves a number of different proteins, including poly(ADP-ribose) polymerase (PARP), DNA-dependent protein kinase, protein kinase C and actin, is important for the initiation of apoptosis. Both substrates can be used to continuously measure the activity of caspase-3.

2. Peroxidases

Reactive oxygen species, including singlet oxygen, superoxide, hydroxyl radical and various peroxides (ROOR′) and hydroperoxides (ROOH) are produced during a number of physiological processes. Activated oxygen species react with a large variety of easily oxidizable cellular components, including NADH, NADPH, dopa, ascorbic acid, histidine, tryptophan, tyrosine, cysteine, glutathione, proteins and nucleic acids. Reactive oxygen species can also oxidize cholesterol and unsaturated fatty acids, causing membrane lipid peroxidation. The importance of the nitric oxide radical enzyme producer and other reactive oxygen species as biological messengers has been increasingly recognized during the last several years. Assaying of oxidative activity in live cells can be done by using Leuco Dyes. Fluorescein, rhodamine and various other dyes can be chemically reduced to colorless, non-fluorescent leuco dyes. These “dihydro” derivatives are readily oxidized back to the parent dye by some reactive oxygen species and thus can serve as fluorogenic probes for detecting oxidative activity in cells. Dihydroethidium, dichlorodihydrofluorescein (H2DCF) and dihydrorhodamine 123 react with intracellular hydrogen peroxide—a reaction mediated by peroxidase, cytochrome C or Fe²⁺. The leuco dyes also serve as fluorogenic substrates for peroxidase enzymes.

3. Glucose Oxidase

The enzyme glucose oxidase is widely used for glucose determination. Glucose oxidase reacts with glucose to form gluconolactone and H₂O₂. The H₂O₂ is then detected using fluorescent probe as described above.

4. Carbonic Anhydrase

Carbonic anhydrase catalyzes the reversible hydration of CO₂ to carbonic acid. Acetazolamide has been shown to bind carbonic anhydrases in a wide variety of eulkaryotic cells. Fluorescent-labeled derivative of acetazolamide is used for studying carbonic anhydrase activity in live cells.

As was described hereinabove, a major embodiment of present invention involves the measurement in individual cells of K_{MAPP} and V_{MAX} values of particular cellular enzymes. This is a rather important assay relating to drug activity within a single intact cell.

In general, pre drug-treated cells are exposed to at least 2 different substrate concentrations in order to determine the enzymatic K_{MAPP} and V_{MAX} values. The same cells are then exposed to the investigated drug (or any other biologically active material, such as, inducer, inhibitor,etc.), during a selected period of time. Finally, the cells are again exposed either to the same 2 substrate concentrations or another 2 or more substrate concentrations, and the K_{MAPP} V_{MAX} values of the drug-treated cells, is determined.

In the following, an example is given in order to demonstrate this principle. Peripheral blood lymphocytes were loaded on a CC, and exposed to FDA, after which individual FI(t) was measured. The same trapped cells, on the same CC were then rinsed (R) twice with fresh buffer and incubated at 37° C. in the presence of hydrogen peroxide (an apoptotic inducer). At the end of incubation, the same cells were again exposed to the same FDA concentration and FI(t) measurements were again performed.

Despite the fact that such an experimental procedure is self consistent (since it has its own control on an individual cell basis, namely control measurements of K_{MAPP} and V_{MAX} of cells, prior to their incubation with the drug), an additional experiment was carried out as a second external control, but this time cells were incubated without the drug.

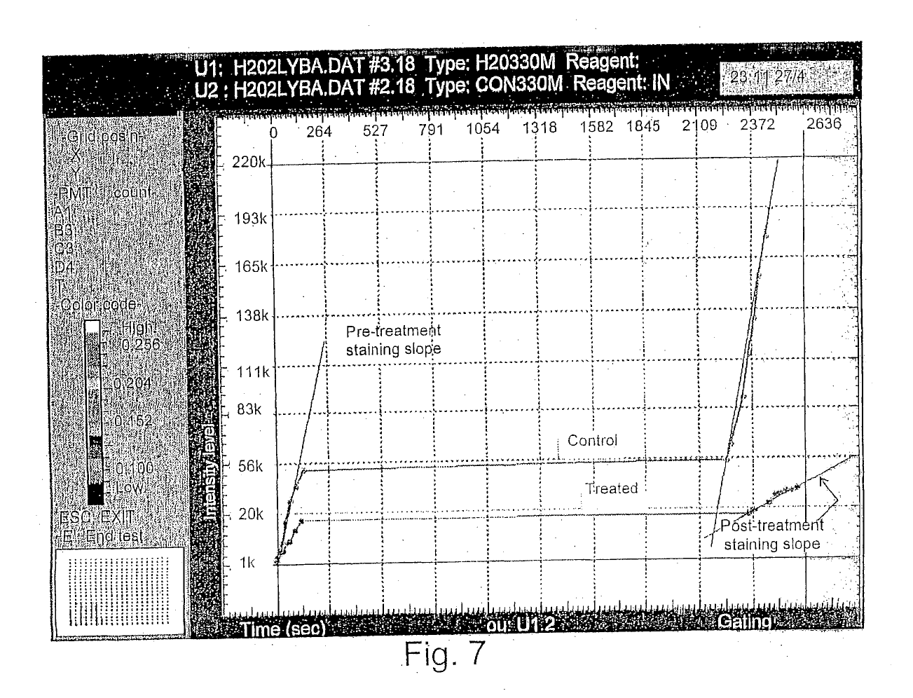

FI(t) of two representative cells, measured prior to and after incubation with (treated) and without (control) hydrogen peroxide (the drug) are shown in FIG. 7. Since cells are in general heterogeneous, one would expect a distribution of FI(t) rates (slopes) in the same experiment. This is why the initial slopes (V₀) of the two curves in FIG. 7 are not identical. Thus, in such an experimental procedure, the determining parameter is the ratio between the initial and the final slopes, namely, the ratios between FI(t) slopes prior to and after incubation (with and without drugs), as well as ratios of individual K_{MAPP} and V_{MAX} prior to and after incubation.

Calculation of both slope ratios shown in FIG. 7 indicates that exposure of lymphocytes to mild oxidative stress resulted in a lower rate of the second staining reaction, in comparison to control. The ratio between the first and the second reactions reflected the apoptotic activity of the inducer Moreover, it can provide an idea regarding apoptotic resistance of specific individual cells.

1. A process for measuring enzymatic activity in an identified, isolated, intact, single, viable cell, comprising the steps:
(a) placing each of the viable cells within individual identified locations on a carrier of a cytometer having means to measure enzymatic activity of a single viable cell placed in an identified location,
(b) exposing the identified isolated cell to a substrate of an enzyme to be measured, and
(c) measuring the rate of product formed or released following every exposure of the cell to same or different concentrations of the substrate.

2. A process according to claim 1, wherein the isolated cell is exposed to a sequence of at least two different concentrations of the substrate and for each exposure the rate of product formed or released, is measured.

3. A process according to claim 2 for measuring the kinetic of a particular enzyme, wherein initial rate production (Vₒ-velocities) are measured from which V_{MAX} and K_{M} are calculated.

4. A process according to claim 1, wherein activities of several different enzymes are measured in the same isolated cell in a population.

5. A process according to claim 1, wherein activity of a particular enzyme is measured before and after the treatment of said isolated cell with a biologically active material.

6. A process according to claim 5, wherein the biologically active material is a drug.

7. A process according to claim 5, wherein the biologically active material is an inhibitor of any of the treated cell's functions.

8. A process according to claim 5, wherein the biologically active material stimulates, induces or promotes a particular function or property of the treated cell.

9. A process according to claim 5, wherein the production rates (Vₒ) are measured and V_{MAX} and K_{M} are calculated before and after cell treatments.

10. A process according to claim 1, wherein the substrate consists of a known fluorescent substance that as a result of enzymatic activity is converted into a measurable fluorescentic product.

11. A process according to claim 10, wherein the substrate is fluorescein-diacetate (FDA).

12. A process according to claim 1, wherein the measured activity is of an intra-cellular enzyme.

13. A process according to claim 12, wherein the intra-cellular enzyme is selected from the group comprising esterase, protease, peptidase, peroxidase, glucose oxidase and carbonic anhydxase.

14. A process according to claim 1, wherein the measured activity is of an extra-cellular enzyme.

15. A process according to claim 1 wherein the isolated single cell is a lymphocyte.

16. A process according to claim 1, wherein the isolated single cell is a lymphocyte, the enzyme is an esterase and the substrate is fluorescein-diacetate.

17. A process according to claim 1, wherein the substrate is color-less and the product formed or released is colored.

18. A process according to claim 1, wherein the substrate is colored and the product formed or released is color-less.
